(19) United States
(12) Patent Application Publication
(10) Pub. No.: US 2024/0312590 A1
(43) Pub. Date: Sep. 19, 2024

(54) SYSTEMS AND METHODS FOR PERFORMING TITRATION OF BASAL AND BOLUS INSULIN

(71) Applicant: Sanofi, Paris (FR)

(72) Inventor: Swati Totawat, Lexington, MA (US)

(21) Appl. No.: 18/576,846

(22) PCT Filed: Jul. 18, 2022
(86) PCT No.: PCT/US2022/037442

§ 371 Date: Jan. 5, 2024

Related U.S. Application Data
(60) Provisional application No. 63/223,429, filed on Jul. 19, 2021.

(30) Foreign Application Priority Data
Oct. 27, 2021 (EP) 21315203.6

(51) Int. Cl. G16H 20/17; A61M 5/172

(52) U.S. Cl. CPC G16H 20/17; A61M 5/1723; A61M 2202/07; A61M 2205/502; A61M 2230/201

(57) ABSTRACT

A system for providing dose recommendations of a first and second type of insulin to a user device is described. The system includes a data storage configured to store a medical prescription, measurement values of the blood glucose level, and dose values of the first type of insulin; and one or more processors coupled to the data storage and configured to determine a next dose value of the first type of insulin based on the measurement values, the dose values of the first type of insulin, and dose adjustment rules specified by the prescription: cause a display, on a user interface of the user device, the next dose value of the first type of insulin: determine a next dose value of the second type of insulin based on the dose maintenance rule; and cause a display, on the user interface, the next dose value of the second type of insulin.

### CROSS REFERENCE TO RELATED APPLICATIONS

This application is a non-provisional of and claims priority to U.S. Provisional Patent Application No. 63/233,429, filed on Jul. 19, 2021, and EP patent application Ser. No. 21/315,203.6, filed on Oct. 27, 2021, the entire contents of which are hereby incorporated by reference.

### BACKGROUND

Diabetes mellitus is a group of metabolic diseases in which a person has high blood sugar, either because the pancreas does not produce enough insulin or because cells do not respond to the insulin that is produced. The treatment of diabetes concentrates on keeping blood sugar levels as close to normal (“euglycemia”) as possible without causing hypoglycemia. This can usually be accomplished with diet, exercise, and use of appropriate medications, for example, insulin in the case of type 1 diabetes and oral medications, as well as possibly insulin, in type 2 diabetes.

Insulin therapy is a common diabetes treatment and an effective way to lower blood sugar levels. Management of diabetes with insulin typically includes periodic checks of the glucose concentration in the blood performed by the patients themselves, in order to obtain regular information on the progress and success of a prescribed treatment. The patient participation is vital since the complications of diabetes are far less common and less severe in patients who have well-managed blood sugar levels. The blood glucose level of the patients may fluctuate throughout the day as it is directly influenced by the amount of insulin administered, as well as lifestyle factors such as the amount and kind of food that is consumed, the exercise level and stress.

### SUMMARY

In general, one innovative aspect of the subject matter described in this specification can be embodied in a system for providing dose recommendations of a first type of insulin and a second type of insulin to a computing device of a patient. The system includes a data storage configured to store: a medical prescription specifying a dose plan for stabilizing a blood glucose level of a patient, the dose plan comprising a plurality of dose adjustment rules for adjusting a dose of a first type of insulin, and a dose maintenance rule for maintaining a dose of a second type of insulin. A plurality of measurement values of the blood glucose level are generated by a blood glucose measurement unit, and a plurality of dose values of the first type of insulin are applied before the plurality of measurement values are generated. The system includes one or more processors operatively coupled to the data storage, in which the one or more processors are configured to: determine a next dose value of the first type of insulin based on the plurality of measurement values of the blood glucose level, the plurality of dose values of the first type of insulin, and the plurality of dose adjustment rules, cause a display, on a user interface of a computing device, of the next dose value of the first type of insulin, determine a next dose value of the second type of insulin based on the dose maintenance rule, and cause a display, on the user interface, of the next dose value of the second type of insulin.

Another innovative aspect of the subject matter described in this specification can be embodied in a method for providing dose recommendations of a first type of insulin and a second type of insulin to a computing device of a patient. The method includes retrieving, from a data storage, input data including: a medical prescription specifying a dose plan for stabilizing a blood glucose level of a patient, the dose plan comprising a plurality of dose adjustment rules for adjusting a dose of a first type of insulin, and a dose maintenance rule for maintaining a dose of a second type of insulin. A plurality of measurement values of the blood glucose level are generated by a blood glucose measurement unit, and a plurality of dose values of the first type of insulin are applied before the plurality of measurement values are generated. The method further includes determining a next dose value of the first type of insulin based on the plurality of measurement values of the blood glucose level, the plurality of dose values of the first type of insulin, and the plurality of dose adjustment rules, and causing a display, on a user interface of a computing device, of the next dose value of the first type of insulin. The method includes determining a next dose value of the second type of insulin based on the dose maintenance rule, and causing a display, on the user interface, of the next dose value of the second type of insulin.

Other embodiments of this aspect include corresponding computer systems, apparatus, and computer programs recorded on one or more computer storage devices, each configured to perform the actions of the method. A system of one or more computers can be configured to perform particular actions by virtue of having software, firmware, hardware, or a combination of them installed on the system that in operation causes or cause the system to perform the actions. One or more computer programs can be configured to perform particular actions by virtue of including instructions that, when executed by data processing apparatus, cause the apparatus to perform the actions.

The foregoing and other embodiments can each optionally include one or more of the following features, alone or in combination. In some embodiments, the first type of insulin is basal insulin and the second type of insulin is bolus insulin. The plurality of measurement values of the blood glucose level may include a plurality of fasting blood glucose values. The first type of insulin may be bolus insulin and the second type of insulin may be basal insulin. The plurality of measurement values of the blood glucose level may include a plurality of postprandial blood glucose values. The plurality of dose adjustment rules may define a target range of blood glucose level. The target range may include a lower limit and an upper limit.

Determining a next dose value of the first type of insulin based on the plurality of measurement values of the blood glucose level, the plurality of dose values of the first type of insulin, and the plurality of dose adjustment rules may include: determining. based on a most recently generated measurement value in the plurality of measurement values of the blood glucose, whether the most recently generated measurement value is within the target range, below the lower limit or above the upper limit, wherein the most recently generated measurement value was generated after a most recently applied dose value in the plurality of dose values of the first type of insulin was applied; and determining the next dose value of the first type of insulin based on the determining of whether the most recently generated measurement value is within the target range, below the lower limit or above the upper limit in accordance with the plurality of dose adjustment rules.

Determining the next dose value of the first type of insulin based on the determining of whether the most recently generated measurement value is within the target range, below the lower limit or above the upper limit may include: in response to the determining that the most recently generated measurement value is within the target range, setting the most recently applied dose value of the first type of insulin as the next dose value of the first type of insulin: in response to the determining that the most recently generated measurement value is above the upper limit of the target range, determining the next dose value of the first type of insulin by increasing the most recently applied dose value of the first type of insulin by a predetermined amount of insulin or by a predetermined percentage of the most recently applied dose value: or in response to the determining that the most recently generated measurement value is below the lower limit of the target range, determining the next dose value of the first type of insulin by decreasing the most recently applied dose value of the first type of insulin by a predetermined amount of the first type of insulin or by a predetermined percentage of the most recently applied dose value.

The one or more processors may be further configured to: determine whether a hypoglycemic event has occurred based on the plurality of measurement values: in response to the determining that the hypoglycemic event has occurred: cause a display, on the user interface, a prompt to the patient to contact a healthcare provider. Determining whether the hypoglycemic event has occurred may include determining whether at least one of the plurality of measurement values is below a predetermined hypoglycemic blood glucose threshold.

The subject matter described in this specification can be implemented in particular embodiments so as to realize one or more of the following technical advantages.

The techniques described in this specification allow a computing system to dynamically and accurately determining next dose values of one or more types of insulin for a patient according to one or more treatment regimens included in a prescribed dose plan, based on real-time data such as measurement values of blood glucose level and dose values provided by the patient.

In particular, the computing system provides a health care provider (HCP) application that allows a HCP to prescribe a dose plan for stabilizing a blood glucose level of a patient. The dose plan can include one or more treatment regimens corresponding to one or more types of insulin. The computing system allows the HCP to access and monitor the patient's entered blood glucose measurement values and dose values in real-time and send a modified dose plan to the patient's device as necessary. The computing system further provides, through a mobile/web application, the patient with recommendations of the next appropriate dose(s) of insulin (e.g., basal and/or bolus insulin) accordingly to the prescribed dose plan. Through the application, the computing system provides the patient with automatic reminders to measure Fasting Blood Glucose (FBG) levels and/or Postprandial Blood Glucose (PBG) levels based on the prescribed dose plan. Based on these FBG and PBG measurement values, the computing system automatically calculates the next dose(s) of insulin based on a plurality of dose adjustment rules and dose maintenance rules included in the prescribed dose plan. The computing system provides the patient with automatic reminders to administer injections of the one or more types of insulin (e.g., basal and/or bolus insulin).

Therefore, in comparison to many existing systems which do not take into account real-time data, such as measurement values of blood glucose level and dose values provided by the patient, systems that implement the described techniques can help patients to achieve better glycemic control by (i) allowing HCPs to prescribe a more complex dose plan that involves multiple types of insulin and to easily modify the prescribed dose plan as necessary, and (ii) providing the patients with more timely and more accurate next dose recommendations (compared to next dose recommendations produced by existing systems) by dynamically calculating the next doses based on patient-provided real-time data and the dose adjustment/maintenance rules described in the prescribed dose plan.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example of a computing environment including a data processing system for performing titration of one or more types of insulin.

FIGS. 2-8 illustrate example screens of a HCP application.

FIGS. 9-12 illustrate example screens of a patient application.

FIG. 13 is a flow diagram of an example process for providing dose recommendations of a first and second type of insulin to a user device.

Like reference numbers and designations in the various drawings indicate like elements.

### DETAILED DESCRIPTION

This specification describes systems and methods for performing titration of one or more types of insulin and providing next dose recommendations to a user device of a patient.

Diabetes mellitus is a group of metabolic diseases in which a person has high blood sugar, either because the pancreas does not produce enough insulin or because cells do not respond to the insulin that is produced. The treatment of diabetes concentrates on keeping blood sugar levels as close to normal (“euglycemia”) as possible without causing hypoglycemia. This can usually be accomplished with diet, exercise, and use of appropriate medications, for example, insulin in the case of type 1 diabetes and oral medications, as well as possibly insulin, in type 2 diabetes.

Insulin therapy is a common diabetes treatment and an effective way to lower blood sugar levels. Essential elements of the management of diabetes with insulin are periodic checks of the glucose concentration in the blood performed by the patients themselves, in order to obtain regular information on the progress and success of a prescribed treatment. The patient participation is vital since the complications of diabetes are far less common and less severe in patients who have well-managed blood sugar levels. The blood glucose level of patients may fluctuate throughout the day as it is directly influenced by the amount of insulin administered, as well as lifestyle factors such as the amount and kind of food that is consumed, the exercise level and stress. For good or perfect glycemic control, the dose of insulin needs to be adjusted for each patient based on measurement values of the patient's blood glucose levels in order to achieve a target blood glucose level. Adjusting a dose of insulin based on measurement values of a patient's blood glucose level is referred to as “titration” of insulin.

There are mainly two types of blood glucose measurement values: fasting blood glucose (FBG) measurement values and postprandial blood glucose (PBG) measurement values.

A fasting blood glucose measurement value is derived after several hours without eating (6 to 8 hours). The fasting blood glucose measurement value is typically taken in the morning before breakfast and is the most commonly performed test among insulin treated patients as it is used to assess the quality of the titration of long-acting insulin (e.g., basal insulin or insulin glargine). The advantage of long-acting insulin is that they have a longer duration of action (e.g., more than 24 hours or even more) compared to the duration of action offered by intermediate-acting insulin. Thus, the profile more closely resembles the basal insulin secretion of the normal pancreatic B-cells.

A postprandial blood glucose measurement value is derived after a meal. Because the absorption of food persists for about 5-6 hours after a meal in both diabetic and nondiabetic individuals, the optimal time to measure postprandial glucose concentration can be determined for each individual. In general, a measurement of postprandial blood glucose 2 hours after the start of a meal is practical, generally approximates the peak value in patients with diabetes, and provides a reasonable assessment of postprandial hyperglycemia. Specific clinical conditions, such as gestational diabetes or pregnancy complicated by diabetes, may benefit from testing at 1 hour after the meal. For diabetes patients who are receiving fast-acting insulin treatments, postprandial blood glucose measurement value are used to assess the quality of the titration of the fast-acting insulin (e.g. bolus insulin).

To achieve good glycemic control, the doses of insulin need to be frequently adjusted for each individual based on the most recent measurement values of the individual's blood glucose level. However, in practice, such insulin titration is challenging for many primary healthcare providers (HCPs) and patients. Physician-managed titration requires close patient monitoring and regular clinic visits or contact between HCPs and patients. Unfortunately, clinic visits or contact can be infrequent due to low patient awareness of the need for follow-up and due to a lack of effective tools for HCPs and patients to communicate with each other. Patients can be trained to do their own titration, however, many patients are unable or unwilling to self-titrate since it is not easy for them to keep track of all measurement values of their blood glucose level and corresponding dose values that have been applied, let alone calculating the next dose based on a dose plan. The complexity of a dose plan increases if the dose plan includes multiple treatment regimens corresponding to multiple types of insulin, making it even more challenging for patients to self-titrate. These patient and clinician factors can contribute to unintentional dosing errors and delays in increasing or decreasing the amount of insulin in treatment regimens. Thus, despite the introduction of advanced insulin delivery devices, many patients with diabetes continue to experience suboptimal glycemic control due to the lack of effective tools for insulin titration/management.

The techniques described in this specification address some of the above challenges by allowing a computing system to dynamically and accurately determine next dose values of one or more types of insulin for a patient according to one or more treatment regimens described in a prescribed dose plan, in response to real-time data such as measurement values of blood glucose level and dose values provided by the patient.

In particular, the computing system allows a HCP to prescribe, through a HCP application, a dose plan for stabilizing a blood glucose level of a patient, in which the dose plan can include multiple treatment regimens corresponding to multiple types of insulin. The computing system allows the HCP to access and monitor the patient's entered blood glucose measurement values and dose values in real-time and send a modified dose plan to the patient's device as necessary. The computing system further provides, through a mobile/web application, the patient with recommendations of the next appropriate dose(s) of insulin (e.g., basal and/or bolus insulin) accordingly to the prescribed dose plan. Through the application, the computing system provides the patient with automatic reminders to measure Fasting Blood Glucose (FBG) levels and/or Postprandial Blood Glucose (PBG) levels based on the prescribed dose plan. Based on these FBG and PBG measurement values, the computing system automatically calculates the next dose(s) of insulin based on a plurality of dose adjustment rules and dose maintenance rules described in the prescribed dose plan. The computing system provides the patient with automatic reminders to administer injections of the one or more types of insulin (e.g., basal and/or bolus insulin).

Therefore, in comparison to many existing systems, systems that implement the described techniques can help patients to achieve better glycemic control by allowing HCPs to prescribe a more complex dose plan that involves multiple types of insulin and to easily modify the prescribed dose plan as necessary, and by providing the patients with more timely and more accurate next dose recommendations by dynamically calculating the next doses based on patient-provided real-time data and the dose adjustment/maintenance rules described in the prescribed dose plan.

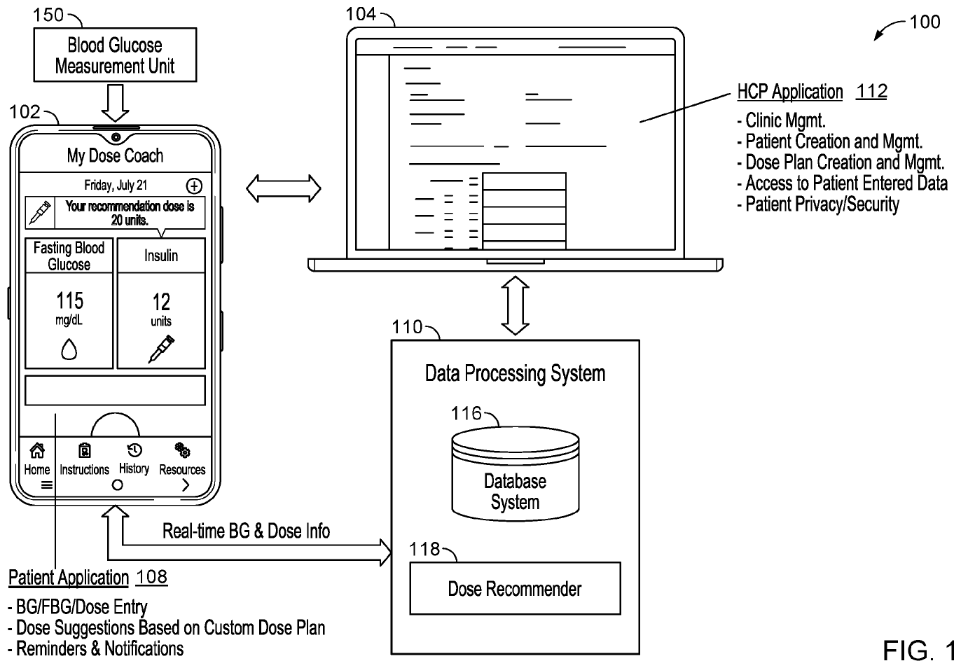

FIG. 1 shows an example of a computing environment for performing titration of one or more types of insulin and providing next dose recommendations to a user device of a patient.

The computing environment 100 includes a data processing system 110, a blood glucose measurement unit 150, a first user device 102 of a patient having a patient application 108 installed on the device 102, and a second user device 104 of a healthcare provider (HCP) having a HCP application 112 installed on the device 104. The patient application 108 and HCP application 112 can be a mobile application or a web application. The data processing system 110 is an example of a server system implemented as computer programs on one or more computers in one or more locations, in which the systems, components, and techniques described below can be implemented.

The data processing system 110 includes a database system 116 and a dose recommender 118. The database system 116 is configured to store patient data including, for example, a medical prescription received from the second user device 104, blood glucose measurement values of the patient and dose values of insulin applied to the patient before the measurement values are generated. In some implementations, the database system 116 resides at a local server system (e.g., a server system of a hospital or a medical center). In some other implementations, the database system 116 is a cloud-based database system. The dose recommender 118 is configured to determine a next dose value of insulin for the patient based on the measurement values of the blood glucose level, the dose values of insulin applied and the prescribed dose plan. The dose recommender 118 then causes a display of a next dose recommendation including the next dose value and time to administer the next dose on the patient application 108. The dose recommender 118 is an engine that is implemented as one or more software modules or components, installed on one or more computers in one or more locations. In some cases, one or more computers will be dedicated to a particular engine: in other cases, multiple engines can be installed and running on the same computer or computers. While FIG. 1 illustrates the dose recommender 118 separate from the device 102, in some implementations, the dose recommender 118 can reside in the device 102. That means, in some implementations, the dose recommender 118 can be a component of the device 102 and configured to compute a next dose recommendation based on blood glucose measurement values and dose values and/or other data that the patient enters in the patient application, and based on the HCP's prescribed dose plan. In some other implementations, the dose recommender 118 can be a service hosted on a cloud computing system and the next dose computation is performed on the cloud computing system.

The first user device 102 and the second user device 104 can communicate with the data processing system 110 and communicate with each other via a communication network (not shown). Each of the first user device 102 and second user device 104 can include any appropriate type of computing device such as a desktop computer, a laptop computer, a handheld computer, a tablet computer, a personal digital assistant (PDA), a cellular telephone, a network appliance, a camera, a smart phone, an enhanced general packet radio service (EGPRS) mobile phone, a media player, a navigation device, an email device, a game console, or an appropriate combination of any two or more of these devices or other data processing devices. The communication network can include a large computer network, such as a local area network (LAN), a wide area network (WAN), the Internet, a cellular network, a telephone network (e.g., PSTN) or an appropriate combination thereof connecting any number of communication devices, mobile computing devices, fixed computing devices and server systems.

As shown in the example of FIG. 1, the first user device 102 is a patient's mobile phone having the patient application 108, which is a mobile application, installed on the mobile device. The second user device 104 is a HCP's computer having a HCP application 112. In some implementations, the second user device 104 accesses the HCP application via a web browser. In some other implementations, the second user device 104 installs the HCP application 112 (e.g., as a mobile application). The data processing system 110 coordinates activities between the patient application 102 installed on the mobile device of the patient and the HCP application 112 accessed by the second user device 104 of the HCP.

The HCP application 112 allows the HCP to register a patient who needs a prescribed dose plan for a diabetes treatment, or to find the patient among registered patients. Once the HCP has registered the patient or selected the patient among existing patients, HCP prescribes, through the HCP application 112, a specific dose plan for the patient. The HCP application 112 allows the HCP to prescribe a dose plan that includes one or more treatment regimens corresponding to one or more types of insulin (e.g., basal and/or bolus insulin).

The HCP application 112 then sends the prescribed dose plan to the patient application 108 and the data processing system 110. Optionally, the HCP application 112 can send an activation sequence to the patient application 108 to allow the patient to access/open the patient application 108 the first time.

For example, the HCP configures a dose plan on the HCP application 112 and then clicks on a button, e.g. a “send.” This triggers a message (e.g., an SMS message or email) to be sent to the patient's first user device 102 with a link to the patient application 108, an authentication code/token, and details of the dose plan (the details are hidden inside the message). The patient selects the link on the first user device 102, which takes the patient to perform the activation by entering authentication information (e.g., first name, last name and/or date of birth). If the entered information matches the information associated with authentication code/token, the activation of the patient application 108 is successful. After that, the patient application 108 displays onboarding screens that allow the patient to use the application 108.

The prescribed dose plan is stored in the database system 116. The patient activates the patient application 108 using the activation sequence and reviews the prescribed dose plan. The patient measures blood glucose measurement values using the blood glucose measurement unit 150. The patient records, using the patient application 108, the blood glucose measurement values and dose values that have been applied before the blood glucose measurement values were generated by the unit 150, as per the prescribed dose plan. The patient also records occurrences of a hypoglycemic event. A hypoglycemic event occurs when a measurement value of the patient's blood glucose level is below a predetermined hypoglycemic blood glucose threshold. The hypoglycemic blood glucose threshold can be determined by the HCP.

The patient app 108 sends the dose values, BG measurement values, and hypoglycemia occurrences to the data processing system 110. These dose values, BG measurement values, and hypoglycemia occurrences are stored in the database system 116.

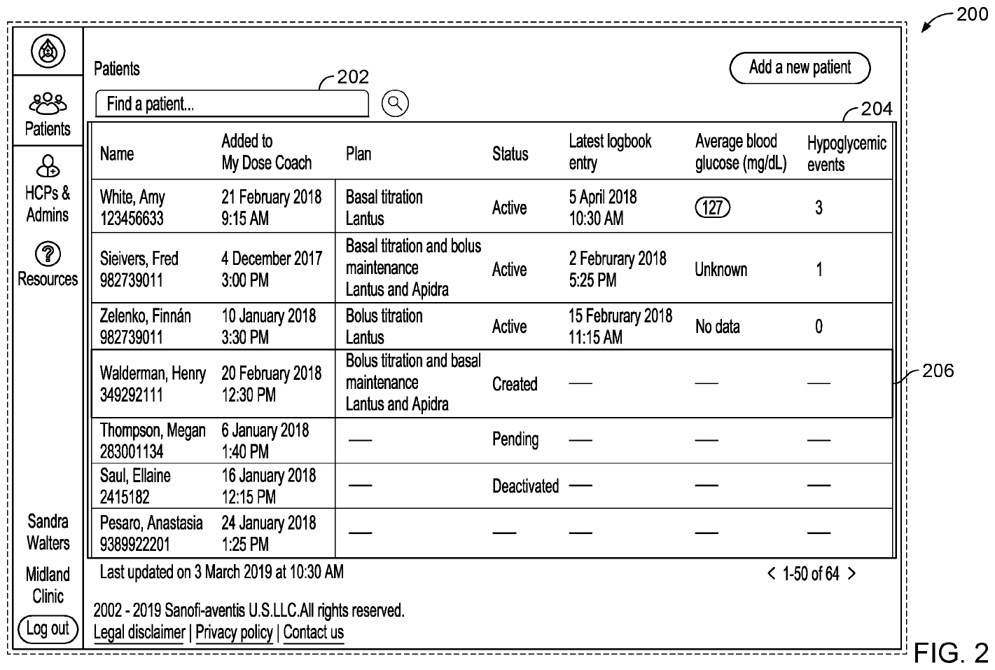

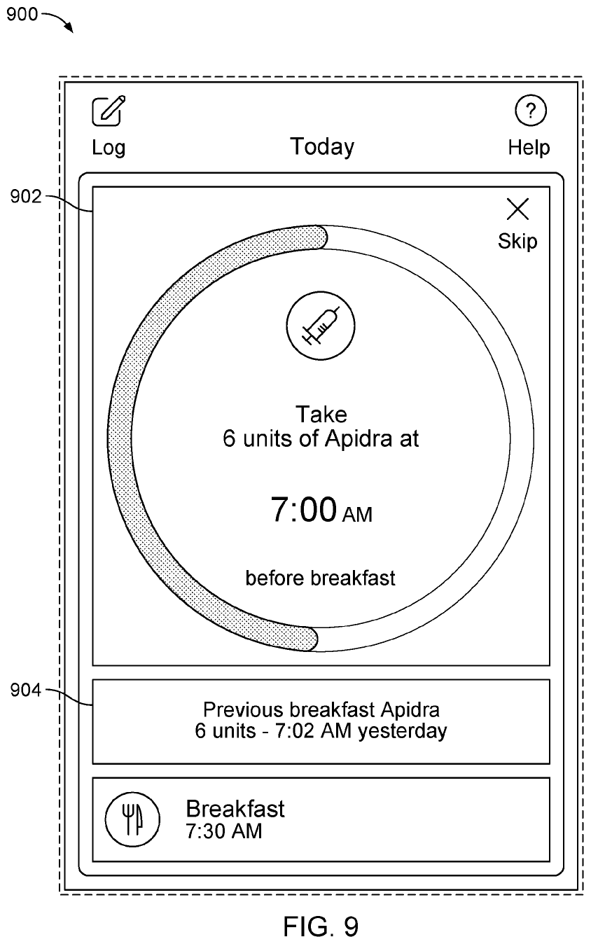

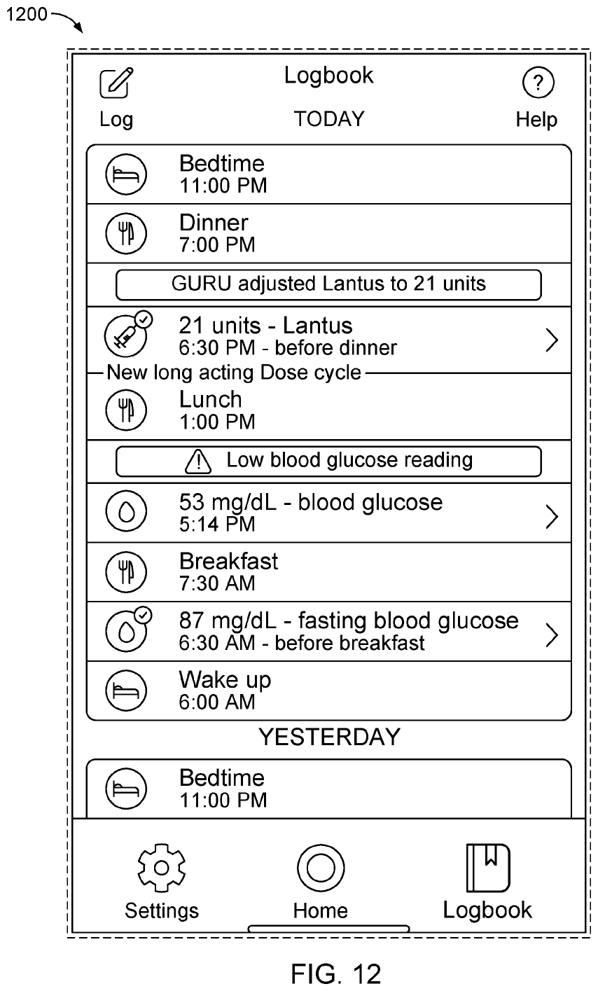

The dose recommender 118 uses the dose plan prescribed by the HCP and provides dose recommendations to the patient application 108 based on the measurement values of the patient's blood glucose level, dose values that have been applied, and hypoglycemia occurrences. The HCP can review the patient-entered data (e.g., dose and BG values) on the HCP application 112 and send a modified dose plan to the patient app, as necessary. The patient can also review the patient-entered data (e.g., dose and BG values) in the patient application. Example screens of the HCP application are shown in FIGS. 2-8, and example screens of the patient application are shown in FIGS. 9-12.

The HCP application 112 allows the HCP to configure a patient-specific dose plan that involves one or more types of insulin. For example, the HCP can configure a patient-specific dose plan from one of the following general plan options:
Basal Titration
Bolus Titration
Basal Titration and Bolus Maintenance
Basal Maintenance and Bolus Titration

Once a general plan is selected, the HCP can then apply modifications to the general plan to generate the patient-specific dose plan based upon the HCP's practice of medicine for individual patient needs.

### Basal Titration Dose Plan (Patients on Basal Insulin Only)

The basal titration dose plan is intended to aid in stabilization of blood glucose levels by adjusting a once-daily dose of long-acting basal insulins.

The basal titration dose plan includes an initial basal dose and basal dose adjustment rules. The initial basal dose specifies a type of insulin drug and its strength, a dose value and time to administer the basal insulin.

For example, the initial basal dose can be specified as follows.
Lantus 100 units/ml
20 units 30 minutes before breakfast daily
Maximum basal dose: 40 units daily

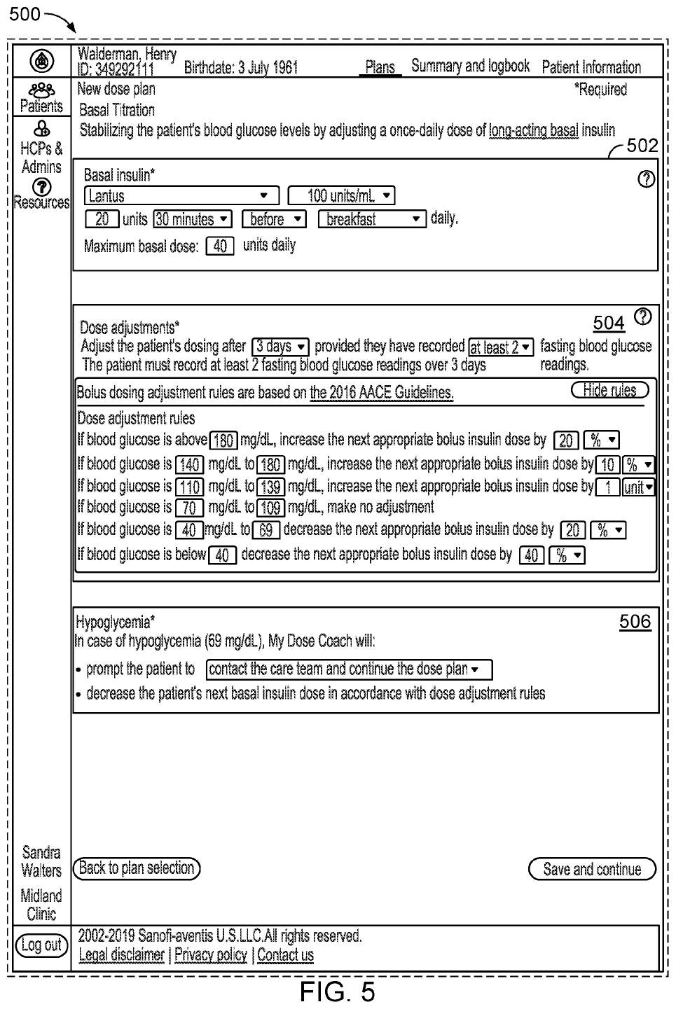

An example of basal dose adjustment rules in a basal titration dose plan is shown in FIG. 5 and described below.
Adjust the patient's dosing after 3 days, provided they have recorded at least 2 fasting blood glucose readings.
If blood glucose is above 180 mg/dL, increase the next appropriate basal insulin dose by 20%.
If blood glucose is 140 mg/dL to 180 mg/dL, increase the next appropriate basal insulin dose by 10%.
If blood glucose is 110 mg/dL to 139 mg/dL, increase the next appropriate basal insulin dose by 1 unit.
If blood glucose is 70 mg/dL to 109 mg/dL, make no adjustment.
If blood glucose is 40 mg/dL to 69 mg/dL, decrease the next appropriate basal insulin dose by 20%.
If blood glucose below 40, decrease the next appropriate basal insulin dose by 40%.
In case of hypoglycemia (69 mg/dL), the patient application will:
   prompt the patient to contact the care team and continue the dose plan
   decrease the patient's next basal insulin dose in accordance with dose adjustment rules.

In particular, when a blood glucose value below a predetermined hypoglycemic BG threshold is entered by the patient, the next basal dose suggestion will be lowered in accordance with the patient's dose plan and the hypoglycemic BG value itself.
A hypoglycemic BG value ensures that a new titration interval is started.
In some implementations, multiple hypoglycemic BG values do not have a cumulative effect on the next dose suggestion. Rather, the lowest hypoglycemic BG value is used to adjust the next dose suggestion. For example, if the last dose suggestion is 20 Units, the patient records a hypoglycemic BG value of 60 mg/dL (for which the dose plan calls for a 2 unit dose reduction), then records a second hypoglycemic BG value of 50 mg/dL (for which the dose plan calls for a 5 unit dose reduction), then the patient's next dose suggestion will be 15 units (20 units minus 5 units).
When a hypoglycemic BG value is entered by the patient, the entry will be evaluated and acted on for the next dose suggestion, regardless of the user-entered measurement time.

### Bolus Titration Dose Plan (Patients on Bolus Insulin Only)

The bolus titration dose plan is intended to aid in stabilization of BG levels by adjusting single or multiple daily doses of mealtime bolus insulin. The HCP uses the HCP application to prescribe the bolus titration dose plan as follows.
HCP selects a type of insulin drug and its strength.
HCP prescribes an initial bolus dose (example default value is 5 units) for each bolus insulin event and the number of the bolus insulin (pre-meal) events (example default value is three events: breakfast, lunch and dinner) that the patient needs to follow.
HCP specifies when the dose adjustments start, e.g., bolus dose adjustments start after
   1-Day or,
   3-Day Average. In this case, the patient application takes the average of the BG values generated during these 3 days to determine the next dose recommendation.
HCP specifies a dose plan duration (example default value is 12 weeks).
HCP configures bolus dose adjustment rules. Example adjustment rules with default values are shown in Table 1. The HCP can adjust these values using the HCP application based upon the practice of medicine.

TABLE 1
Default Bolus Adjustment Values for the Next Titration Interval

| Blood Glucose (mg/dL) at Least 2 Hours | Percentage Change |
|---|---|
| Post-meal and Before Next Meal | in Bolus |

| From | To | Insulin Dose |
|---|---|---|
| 140 and Above | | 10% |
| 70 | 140 | 0 |
| 40 | 60 | −10% |

| | |
|---|---|
| Below 40 | −20% |

In case of a hypoglycemic event, the following actions are suggested to the patient on the patient application:
Continue Dose Plan
Contact Doctor and Continue Dose Plan
Call Doctor and Stop Dose Recommendations

Based on the dose plan prescribed by the HCP, the pre-meal bolus insulin dose increases or decreases according to the BG measurement(s) at least 2 hours after the meal and prior to the next meal obtained during the titration cycle. If the dinner bolus event is prescribed, the bed time BG will be used to titrate the dinner pre-meal bolus insulin of the following day if there is not a 2 hour post-dinner BG measurement.

In the case of a hypoglycemic event following a bolus insulin dose, the patient is asked to take action as per the HCP's instructions. Additionally, the pre-meal bolus insulin dose corresponding to the hypoglycemic event for the next day is reduced as per the adjustment rules, regardless of the selected titration cycle duration.

In particular, if a hypoglycemic BG value is entered by the patient, the pre-meal bolus insulin dose corresponding to the hypoglycemic event for the next day will be lowered in accordance with the patient's dose plan and the hypoglycemic BG value itself.

In some implementations, multiple hypoglycemic BG values do not have a cumulative effect on the next dose suggestion. Rather, the lowest hypoglycemic BG value is used to adjust the next dose suggestion. If multiple hypoglycemic BG values are recorded for a Bolus event, then the lowest hypoglycemic BG value is used to adjust the dose suggestion corresponding to the hypoglycemic event for the next day, in accordance with the patient's dose plan and the hypoglycemic BG value itself.

### Detailed Bolus Titration Example:

Example dose plan: HCP prescribes a Bolus 1-day titration dose plan with starting dose for bolus at 10 units with three bolus injections that needs to be taken at breakfast, lunch and dinner. The hypoglycemic value is configured at 70 mg/dL. The example dose adjustment rules are listed in Table 2.

TABLE 2
Example Dose Adjustment Rules

| Blood Glucose (mg/dL) at Least 2 Hours | Percentage Change |
|---|---|
| Post-meal and Before Next Meal | in Bolus |

| From | To | Insulin Dose |
|---|---|---|
| 140 and Above | | 10% |
| 70 | 140 | 0 |
| 40 | 69 | −10% |

| | |
|---|---|
| Below 40 | −20% |

Applying the example dose plan and example dose adjustment rules, the dose recommender 118 will calculate next doses and provide dose suggestions to the patient application for displaying to the patient, as described in Table 3 below.

TABLE 3
Example Bolus Titration Dose Suggestions

| | Day 1 | Day 2 | Day 3 |
|---|---|---|---|
| Breakfast | Pre-meal BG is 100 mg/dL | Pre-meal BG is 142 mg/dL | Pre-meal BG is 100 mg/dL |
| | Pre-meal bolus insulin | Pre-meal bolus insulin | Pre-meal bolus insulin |
| | dose suggested by app is | dose suggested by app is | suggested by app is 12 |
| | 10 Units | 11 units (increase dose | units (increase dose |
| | Pre-meal bolus insulin | suggestion by 10%, as the | suggestion by 10%, as the |
| | taken by patient is 10 Units | pre-meal BG for lunch on | pre-meal BG for lunch on |
| | Pre-meal bolus insulin | the previous day (Day 1) | the previous day (Day 2) |
| | recorded in app is 10 Units | was 141 mg/dL, which is | was 143 mg/dL, which is |
| | | higher than the target | higher than the target |
| | | range) | range) |
| | | Pre-meal bolus insulin | Pre-meal bolus insulin |
| | | taken by patient is 11 Units | taken by patient is 12 Units |
| | | Pre-meal bolus insulin | Pre-meal bolus insulin |
| | | recorded in app is 11 Units | recorded in app is 12 Units |
| Lunch | Pre-meal BG is 141 mg/dL | Pre-meal BG is 143 mg/dL | Pre-meal BG is 152 mg/dL |
| | Pre-meal bolus insulin | Pre-meal bolus insulin | Pre-meal bolus insulin |
| | suggested by app is 10 | suggested by app is 11 | suggested by app is 12 |
| | Units | Units (increase dose | Units (increase dose |
| | Pre-meal bolus insulin | suggestion by 10%, as the | suggestion by 10%, as the |
| | taken by patient is 10 Units | pre-meal BG for dinner on | pre-meal BG for dinner on |
| | Pre-meal bolus insulin | the previous day (Day 1) | the previous day (Day 2) |
| | recorded in app is 10 Units | was 150 mg/dL, which is | was 144 mg/dL, which is |
| | | higher than the target | higher than the target |
| | | range) | range) |
| | | Pre-meal bolus insulin | Pre-meal bolus insulin |
| | | taken by patient is 11 Units | taken by patient is 12 Units |
| | | Pre-meal bolus insulin | Pre-meal bolus insulin |
| | | recorded in app is 11 | recorded in app is 12 |
| | | Units | Units |
| | recorded in app is 10 Units | higher than the target | higher than the target |
| | | range) | range) |
| | | Pre-meal bolus insulin | Pre-meal bolus insulin |
| | | taken by patient is 11 Units | taken by patient is 12 Units |
| | | Pre-meal bolus insulin | Pre-meal bolus insulin |
| | | recorded in app is 11 Units | recorded in app is 12 Units |
| Lunch | Pre-meal BG is 141 mg/dL | Pre-meal BG is 143 mg/dL | Pre-meal BG is 152 mg/dL |
| | Pre-meal bolus insulin | Pre-meal bolus insulin | Pre-meal bolus insulin |
| | suggested by app is 10 Units | suggested by app is 11 | suggested by app is 12 |
| | Pre-meal bolus insulin | Units (increase dose | Units (increase dose |
| | taken by patient is 10 Units | suggestion by 10%, as the | suggestion by 10%, as the |
| | Pre-meal bolus insulin | pre-meal BG for dinner on | pre-meal BG for dinner on |
| | recorded in app is 10 Units | the previous day (Day 1) | the previous day (Day 2) |
| | | was 150 mg/dL, which is | was 144 mg/dL, which is |
| | | higher than the target | higher than the target |
| | | range) | range) |
| | | Pre-meal bolus insulin | Pre-meal bolus insulin |
| | | taken by patient is 11 | taken by patient is 12 |
| | | Units | Units |
| | | Pre-meal bolus insulin | Pre-meal bolus insulin |
| | | recorded in app is 11 | recorded in app is 12 |
| | | Units | Units |
| Dinner | Pre-meal BG is 150 mg/dL | Pre-meal BG is 144 mg/dL | Pre-meal BG is 170 mg/dL |
| | Pre-meal bolus insulin | Pre-meal bolus insulin | Pre-meal bolus insulin |
| | suggested by app is 10 | suggested by the app is | suggested by app is 11 |
| | Units | 11 Units (increase dose | Units (keep dose |
| | Pre-meal bolus insulin | suggestion by 10%, as the | suggestion the same, as |
| | taken by patient is 10 Units | bedtime BG for the | the bedtime BG for the |
| | Pre-meal bolus insulin | previous day (Day 1) was | previous day (Day 2) was |
| | recorded in app is 10 Units | 153 mg/dL, which is higher | 100 mg/dL, which is in |
| | | than the target range) | target range) |
| | | Pre-meal bolus insulin | Pre-meal bolus insulin |
| | | taken by patient is 11 Units | taken by app is 11 Units |
| | | Pre-meal bolus insulin | Pre-meal bolus insulin |
| | | recorded in app is 11 | recorded in app is 11 |
| | | Units | Units |
| Bed Time BG | Bedtime BG is 153 mg/dL | Bedtime BG is 100 mg/dL | Bedtime BG is 130 mg/dL |

### Basal Titration and Bolus Maintenance Dose Plan

The basal titration and bolus maintenance dose plan is intended to aid in stabilization of BG levels where the HCP has determined it a priority to adjust basal insulin. This plan titrates once-daily doses of long-acting basal insulin while maintaining mealtime bolus insulin doses. The basal titration and bolus maintenance dose plan is configured by the HCP through the HCP application as shown below.
Basal Titration Rules (as shown in section Basal Titration Dose Plan above)
Bolus Maintenance Rules
   Bolus Insulin Drug and strength
   Bolus maintenance dose(s) for the day
   Hypoglycemia action when BG is below Hypoglycemic value

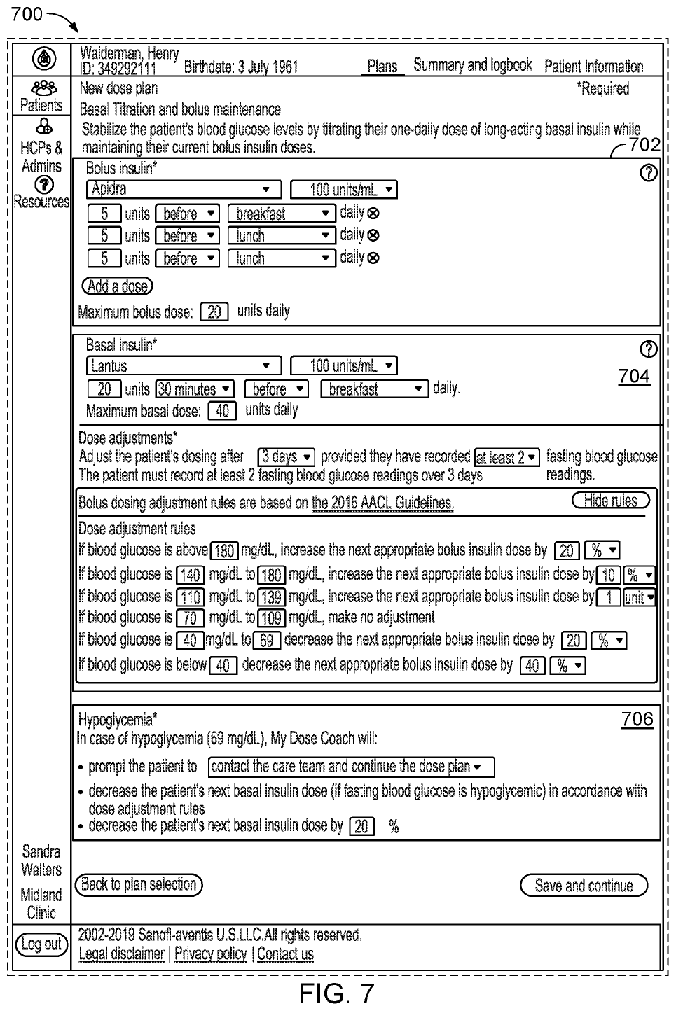

An example of basal titration and bolus maintenance dose plan is shown in FIG. 7.

For this plan, hypoglycemic events will be managed as follows.

When a hypoglycemic BG value is entered by the patient for fasting blood glucose (FBG) and no additional hypoglycemic BG value is entered for the titration day, then the next basal dose will be lowered in accordance with the patient's dose plan and the hypoglycemic BG value.

When a hypoglycemic BG value is entered by the patient for FBG and one or more hypoglycemic BG values are entered for the titration day, then the patient app will “Stop the dose recommendations” and advise the patient to contact the care team for further actions.

When patient's FBG is not in hypoglycemic range, but two or more hypoglycemic BGs following the same bolus event (i.e. breakfast bolus leads to hypo, treat with carbs and get better, but then get hypo again before lunch) are entered, then the patient app will “Stop the dose recommendations” and advise the patient to contact the care team for further actions.

When patient's FBG is not in hypoglycemic range, but two or more hypoglycemic BGs are entered for different bolus events in the day, then the app will “Stop the dose recommendations” and advise the patient to contact the care team for further actions.

When an overnight hypoglycemic BG value (post bedtime BG and pre-FBG/pre-breakfast) is entered, then the patient app will “Stop the dose recommendations” and advise the patient to contact the care team for further actions.

### Bolus Titration and Basal Maintenance Dose Plan

The bolus titration and basal maintenance dose plan is intended to aid in stabilization of BG levels where the HCP has determined it a priority to adjust Bolus insulin. This plan titrates mealtime bolus insulin doses while maintaining the once-daily long-acting Basal insulin dose. The bolus titration and basal maintenance dose plan will be configured by the HCP as follows.
Bolus Titration Rules (as shown in section Bolus Titration Dose Plan above)
Basal Maintenance Rules
   Basal Insulin Drug
   Basal maintenance dose
   Basal dose time
   Hypoglycemia action when FBG is below Hypoglycemic value

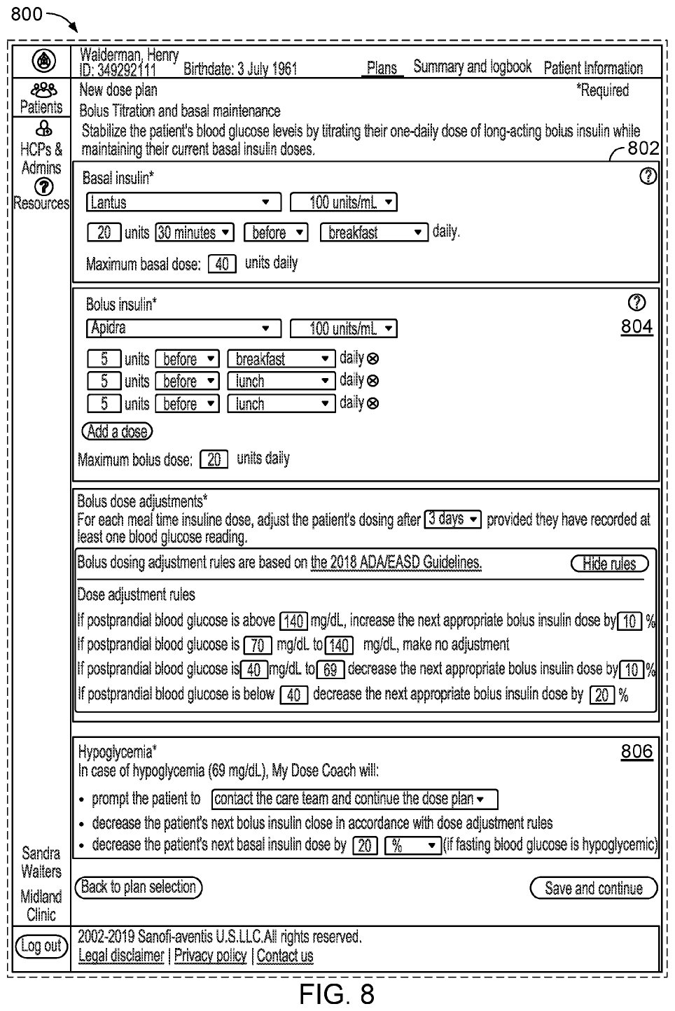

An example of bolus titration and basal maintenance dose plan is shown in FIG. 8

For this plan, hypoglycemic events will be managed as follows.

When a hypoglycemic BG value is entered by the patient for FBG and no additional hypoglycemic BG values are entered for the titration day, then the next basal dose will be lowered in accordance with the patient's dose plan and the hypoglycemic BG value.

When a hypoglycemic BG value is entered by the patient for FBG and one or more hypoglycemic BG values are entered for the titration day, then the app will “Stop the dose recommendations” and advise the patient to contact the care team for further actions.

When patient's FBG is not in hypoglycemic range, but one or more hypoglycemic BGs following the same bolus event are entered (i.e. breakfast bolus leads to hypo, treat with carbs and get better, but then get hypo again before lunch), then the app will reduce the next dose suggestion corresponding to the prior bolus event in accordance with the patient's dose plan and the lowest hypoglycemic BG value.

When patient's FBG is not in hypoglycemic range, but one or more hypoglycemic BGs are entered for a different bolus event that day, then the app will reduce the dose suggestions for the next day bolus events corresponding to each of the hypoglycemic events, in accordance with the patient's dose plan and the hypoglycemic BG value.

When an overnight hypoglycemic BG (post bedtime BG and pre-FBG/pre-breakfast) is entered by the patient, then the app will “Stop the dose recommendations” and advise the patient to contact the care team for further actions.

FIGS. 2-8 illustrate example screens of the HCP application.

As described above, the HCP application allows the HCP to register a patient who needs a prescribed dose plan for a diabetes treatment, or to find the patient among registered patients. FIG. 2 shows an example screen 200 of the HCP application, which allows the HCP to search for a patient by entering the patient's name on box 202, or to select a patient (e.g., patient 206) from a list 204 of registered patient.

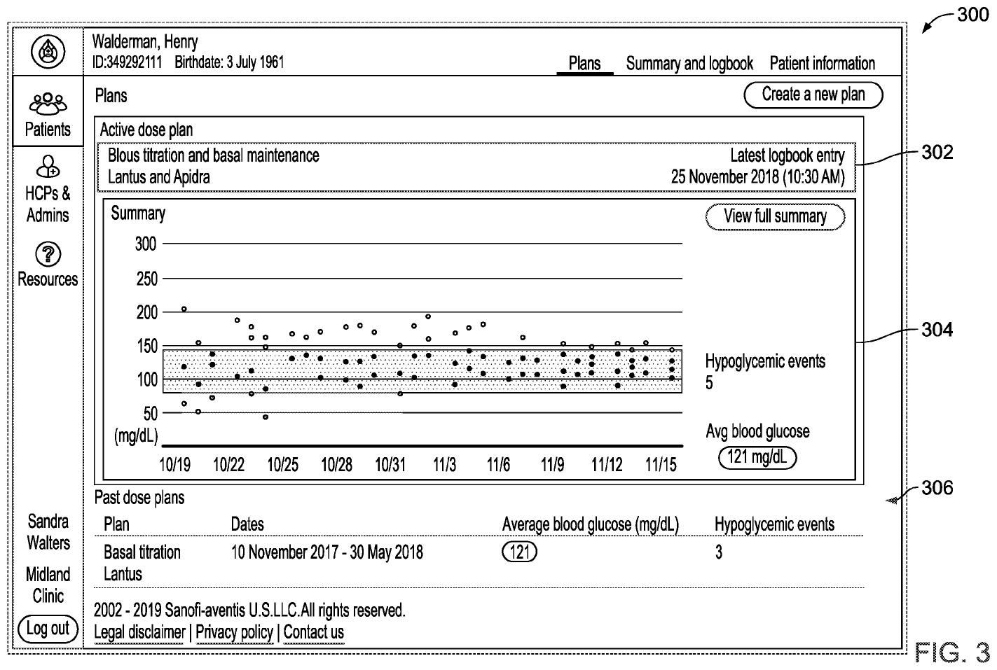

Once the HCP has registered the patient or selected the patient among existing patients, HCP reviews the patient's information and medical history on a patient dashboard displayed on the HCP application. For example, in FIG. 3, the patient dashboard 300 includes an active dose plan 302 that is currently prescribed for the patient, a chart 304 illustrating recent blood glucose measurement values of the patient, and one or more past dose plans 306 that were previously prescribed for the patient.

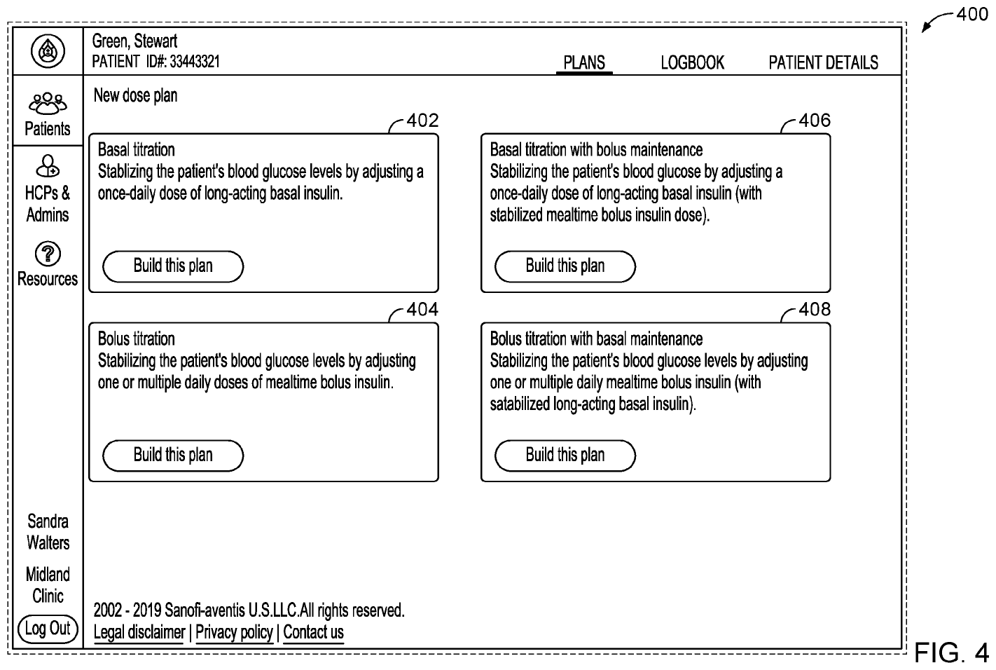

The HCP prescribes, through the HCP application, a specific dose plan for the patient. The HCP application allows the HCP to prescribe a dose plan that includes one or more treatment regimens corresponding to one or more types of insulin (e.g., basal and/or bolus insulin). For example, FIG. 4 shows 4 dose plan options that the HCP can prescribe for the patient: basal titration dose plan 402, bolus titration dose plan 404, basal titration with bolus maintenance dose plan 406, and bolus titration with basal maintenance dose plan 408.

FIG. 5 illustrates an example screen of the HCP application that allows the HCP to configure a basal titration dose plan. The screen 500 includes an area 502 that allows the HCP to configure an initial basal insulin dose, an area 504 that allows the HCP to configure a plurality of basal dose adjustment rules, and an area 506 that allows the HCP to specify actions to be performed by the patient application if a hypoglycemic event occurs.

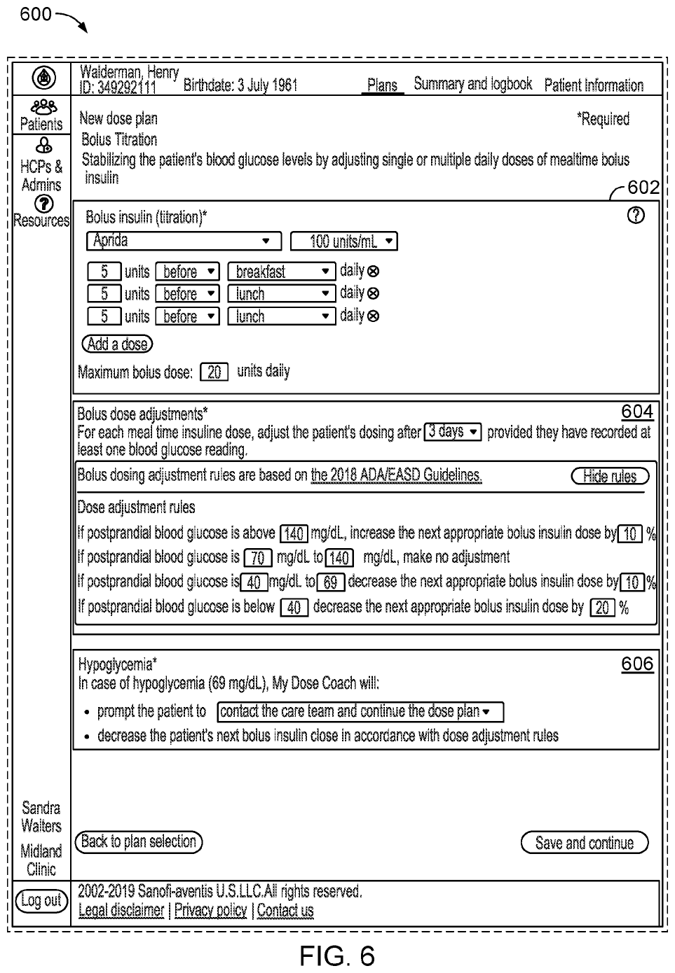

FIG. 6 illustrates an example screen of the HCP application that allows the HCP to configure a bolus titration dose plan. The screen 600 includes an area 602 that allows the HCP to configure initial bolus insulin doses, an area 604 that allows the HCP to configure a plurality of bolus dose adjustment rules, and an area 606 that allows the HCP to specify actions to be performed by the patient application if a hypoglycemic event occurs.

FIG. 7 illustrates an example screen of the HCP application that allows the HCP to configure a basal titration and bolus maintenance dose plan. The screen 700 includes an area 702 that allows the HCP to configure bolus maintenance rules, an area 704 that allows the HCP to configure an initial basal dose and a plurality of basal dose adjustment rules, and an area 706 that allows the HCP to specify actions to be performed by the patient application to adjust the next basal/bolus insulin dose if a hypoglycemic event occurs.

FIG. 8 illustrates an example screen of the HCP application that allows the HCP to configure a bolus otitration and basal maintenance dose plan. The screen 800 includes an area 802 that allows the HCP to configure basal maintenance rules, an area 804 that allows the HCP to configure initial bolus doses and a plurality of bolus dose adjustment rules, and an area 806 that allows the HCP to specify actions to be performed by the patient application to adjust the next basal/bolus insulin dose if a hypoglycemic event occurs.

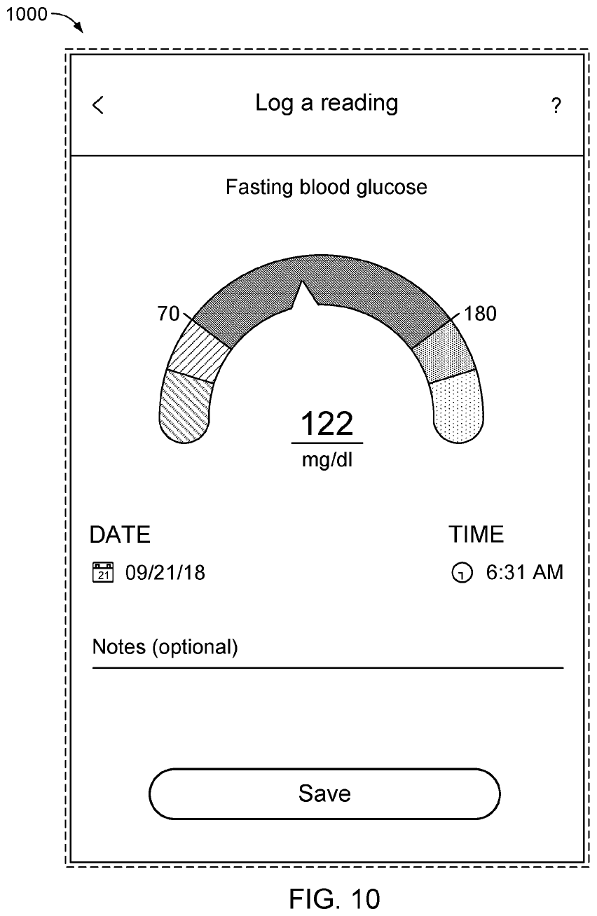

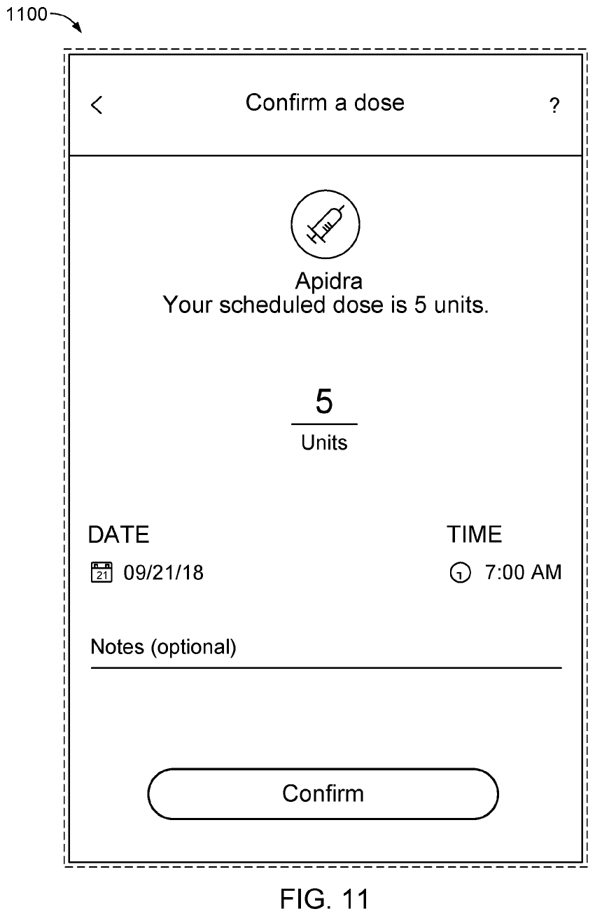

FIGS. 9-12 illustrate example screens of the patient application. The patient application automatically displays the prescribed basal and bolus events in the order in which they are scheduled along with the suggested doses (as shown in the “Today” view). For example, as shown in a screen 900 in FIG. 9, the patient application automatically suggests the next dose of bolus insulin, e.g., 6 units of Apidra, along with the time that the bolus insulin should be administered, e.g., at 7 am before breakfast (902). The patient application can also display a previous dose that was taken at the same time a day before (904). FIG. 10 shows an example screen 1000 of the patient application that allows the user to enter a blood glucose measurement value of the patient's blood glucose level and the time it was measured and then save the reading. FIG. 11 shows an example screen 1100 of the patient application that allows the user to enter an insulin dose value indicating an amount of insulin that was taken for a given event and the time when it was taken and then save the data. As the user enters the blood glucose measurement values and insulin dose values in the patient application, these values are stored in the patient application and can be viewed on a logbook at any point in time. FIG. 1200 illustrates an example screen 1200 of a logbook.

After each of the blood glucose measurement values and insulin dose values is entered in the patient application, the patient application automatically sends the value to the data processing system 110 for storing the value in database system 116. The dose recommender 118 then retrieves the blood glucose measurement values and insulin dose values from the database system 116 and dynamically calculates the next insulin dose based on the prescribed dose plan and the retrieved blood glucose measurement values and insulin dose values. The dose recommender 118 then causes a display of the next insulin dose on the patient application.

The HCP application can access the database system 116 to retrieve the blood glucose measurement values and insulin dose values that have been applied. The HCP can modify the current dose plan based on the retrieved blood glucose measurement values and insulin dose values in order to achieve better glycemic control. For example, the HCP can change a basal titration dose plan to a basal titration and bolus maintenance dose plan. As another example, the HCP can change a basal titration and bolus maintenance dose plan to a bolus titration and basal maintenance dose plan, or vice versa. The HCP application can then send the modified dose plan to the data processing system 110. The dose recommender 118 can dynamically calculate the next dose values of insulin based on the modified dose plan and cause the patient application to display the next doses to the patient.

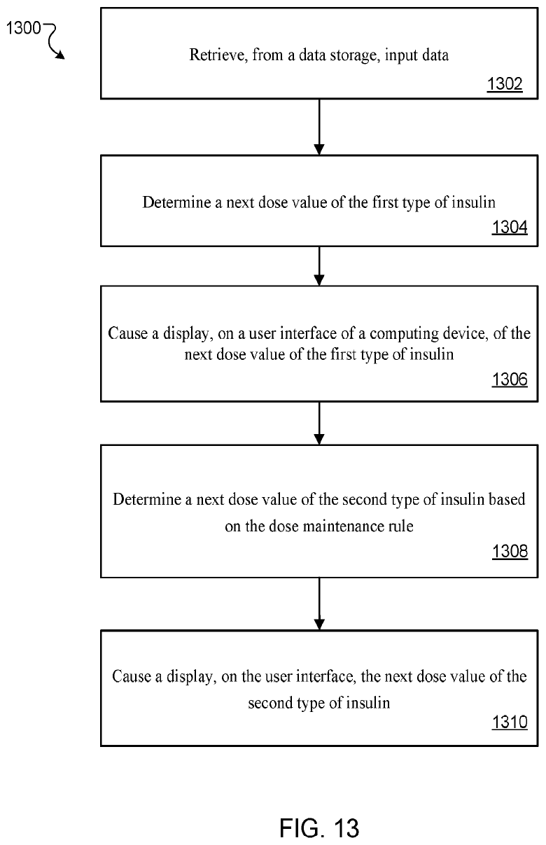

FIG. 13 is a flow diagram of an example process for providing dose recommendations of a first and second type of insulin to a user device. For convenience, the process 1400 will be described as being performed by a system of one or more computers located in one or more locations. For example, a data processing system, e.g., the data processing system 110 of FIG. 1, appropriately programmed in accordance with this specification, can perform the process 1300.

The system retrieves, from a data storage, input data (step 1302). The input data includes: a medical prescription specifying a dose plan for stabilizing a blood glucose level of a patient: a plurality of measurement values of the blood glucose level generated by a blood glucose measurement unit; and a plurality of dose values of the first type of insulin applied before the plurality of measurement values are generated. The dose plan includes (i) a plurality of dose adjustment rules for adjusting a dose of a first type of insulin, and (ii) a dose maintenance rule for maintaining a dose of a second type of insulin. The plurality of dose adjustment rules defines a target range of blood glucose level. The target range includes a lower limit and an upper limit.

In some implementations, the first type of insulin is basal insulin and the second type of insulin is bolus insulin. In these implementations, the plurality of measurement values of the blood glucose level includes a plurality of fasting blood glucose values.

In some other implementations, the first type of insulin is bolus insulin and the second type of insulin is basal insulin. In these implementations, the plurality of measurement values of the blood glucose level comprises a plurality of postprandial blood glucose values.

The system determines a next dose value of the first type of insulin based on the plurality of measurement values of the blood glucose level, the plurality of dose values of the first type of insulin, and the plurality of dose adjustment rules (step 1304). In particular, the system determines, based on a most recently generated measurement value in the plurality of measurement values of the blood glucose, whether the most recently generated measurement value is within the target range, below the lower limit or above the upper limit, wherein the most recently generated measurement value was generated after a most recently applied dose value in the plurality of dose values of the first type of insulin was applied.

In response to the determining that the most recently generated measurement value is within the target range, the system sets the most recently applied dose value of the first type of insulin as the next dose value of the first type of insulin:

In response to the determining that the most recently generated measurement value is above the upper limit of the target range, the system determines the next dose value of the first type of insulin by increasing the most recently applied dose value of the first type of insulin by a predetermined amount of insulin or by a predetermined percentage of the most recently applied dose value.

In response to the determining that the most recently generated measurement value is below the lower limit of the target range, the system determines the next dose value of the first type of insulin by decreasing the most recently applied dose value of the first type of insulin by a predetermined amount of the first type of insulin or by a predetermined percentage of the most recently applied dose value.

The system causes a display, on a user interface of a computing device, of the next dose value of the first type of insulin (step 1306).

The system determines a next dose value of the second type of insulin based on the dose maintenance rule (step 1308).

The system causes a display, on the user interface, of the next dose value of the second type of insulin (step 1310). In some implementations, the system may send a reminder of the next dose of the first type of insulin and/or the second type of insulin to the user device of the user via an additional communication channel. The additional communication channel can be, for example, an email, a short message service (SMS) text message, a multimedia message service (MMS) message, a telephone call, a Voice-over-IP (VOIP) communication, a social media platform notification system, a mobile phone push notification, and other mobile device notification systems.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program can be deployed to be executed on one computer or on multiple computers that are located at one site or distributed across multiple sites and interconnected by a data communication network.

In this specification, the term “database” is used broadly to refer to any collection of data: the data does not need to be structured in any particular way, or structured at all, and it can be stored on storage devices in one or more locations. Thus, for example, the index database can include multiple collections of data, each of which may be organized and accessed differently.

Similarly, in this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine: in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non-volatile memory, media and memory devices, including by way of example semiconductor memory devices, e.g., EPROM, EEPROM, and flash memory devices: magnetic disks, e.g., internal hard disks or removable disks: magneto optical disks; and CD ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well: for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user: for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Embodiments of the subject matter described in this specification can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable sub combination. Moreover, although features may be described above as acting in certain combinations and even initially be claimed as such, one or more features from a claimed combination can in some cases be excised from the combination, and the claimed combination may be directed to a sub combination or variation of a sub combination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

1. A system comprising:
a data storage configured to store:
a medical prescription specifying a dose plan for stabilizing a blood glucose level of a patient, the dose plan comprising (i) a plurality of dose adjustment rules for adjusting a dose of a first type of insulin, and (ii) a dose maintenance rule for maintaining a dose of a second type of insulin,
a plurality of measurement values of the blood glucose level generated by a blood glucose measurement unit, and
a plurality of dose values of the first type of insulin applied before the plurality of measurement values are generated; and
one or more processors operatively coupled to the data storage, the one or more processors configured to:
determine a next dose value of the first type of insulin based on the plurality of measurement values of the blood glucose level, the plurality of dose values of the first type of insulin, and the plurality of dose adjustment rules,
cause a display, on a user interface of a computing device, of the next dose value of the first type of insulin,
determine a next dose value of the second type of insulin based on the dose maintenance rule, and
cause a display, on the user interface, of the next dose value of the second type of insulin.

2. The system of claim 1, wherein the first type of insulin is basal insulin and the second type of insulin is bolus insulin.

3. The system of claim 1, wherein the plurality of measurement values of the blood glucose level comprises a plurality of fasting blood glucose values.

4. The system of claim 1, wherein the first type of insulin is bolus insulin and the second type of insulin is basal insulin.

5. The system of claim 1, wherein the plurality of measurement values of the blood glucose level comprises a plurality of postprandial blood glucose values.

6. The system of claim 1, wherein the plurality of dose adjustment rules define a target range of blood glucose level, the target range comprising a lower limit and an upper limit, and wherein determining a next dose value of the first type of insulin based on the plurality of measurement values of the blood glucose level, the plurality of dose values of the first type of insulin, and the plurality of dose adjustment rules comprises:
determining, based on a most recently generated measurement value in the plurality of measurement values of the blood glucose, whether the most recently generated measurement value is within the target range, below the lower limit or above the upper limit, wherein the most recently generated measurement value was generated after a most recently applied dose value in the plurality of dose values of the first type of insulin was applied; and
determining the next dose value of the first type of insulin based on the determining of whether the most recently generated measurement value is within the target range, below the lower limit or above the upper limit in accordance with the plurality of dose adjustment rules.

7. The system of claim 6, wherein determining the next dose value of the first type of insulin based on the determining of whether the most recently generated measurement value is within the target range, below the lower limit or above the upper limit comprises:
in response to the determining that the most recently generated measurement value is within the target range, setting the most recently applied dose value of the first type of insulin as the next dose value of the first type of insulin;
in response to the determining that the most recently generated measurement value is above the upper limit of the target range, determining the next dose value of the first type of insulin by increasing the most recently applied dose value of the first type of insulin by a predetermined amount of insulin or by a predetermined percentage of the most recently applied dose value; or
in response to the determining that the most recently generated measurement value is below the lower limit of the target range, determining the next dose value of the first type of insulin by decreasing the most recently applied dose value of the first type of insulin by a predetermined amount of the first type of insulin or by a predetermined percentage of the most recently applied dose value.

8. The system of claim 1, wherein the one or more processors are further configured to:
determine whether a hypoglycemic event has occurred based on the plurality of measurement values; and
in response to the determining that the hypoglycemic event has occurred, cause a display, on the user interface, of a prompt to the patient to contact a healthcare provider.

9. The system of claim 8, wherein determining whether the hypoglycemic event has occurred comprises determining whether at least one of the plurality of measurement values is below a predetermined hypoglycemic blood glucose threshold.

10. A computer-implemented method comprising:
retrieving, from a data storage, input data comprising:
a medical prescription specifying a dose plan for stabilizing a blood glucose level of a patient, the dose plan comprising (i) a plurality of dose adjustment rules for adjusting a dose of a first type of insulin, and (ii) a dose maintenance rule for maintaining a dose of a second type of insulin,
a plurality of measurement values of the blood glucose level generated by a blood glucose measurement unit, and
a plurality of dose values of the first type of insulin applied before the plurality of measurement values are generated; and
determining a next dose value of the first type of insulin based on the plurality of measurement values of the blood glucose level, the plurality of dose values of the first type of insulin, and the plurality of dose adjustment rules,
causing a display, on a user interface of a computing device, of the next dose value of the first type of insulin,
determining a next dose value of the second type of insulin based on the dose maintenance rule, and
causing a display, on the user interface, of the next dose value of the second type of insulin.

11. The computer-implemented method of claim 10, wherein the first type of insulin is basal insulin and the second type of insulin is bolus insulin.

12. The computer-implemented method of claim 10, wherein the plurality of measurement values of the blood glucose level comprises a plurality of fasting blood glucose values.

13. The computer-implemented method of claim 10, wherein the first type of insulin is bolus insulin and the second type of insulin is basal insulin.

14. The computer-implemented method of claim 10, wherein the plurality of measurement values of the blood glucose level comprises a plurality of postprandial blood glucose values.

15. The computer-implemented method of claim 10, wherein the plurality of dose adjustment rules defines a target range of blood glucose level, the target range comprising a lower limit and an upper limit, and wherein determining a next dose value of the first type of insulin based on the plurality of measurement values of the blood glucose level, the plurality of dose values of the first type of insulin, and the plurality of dose adjustment rules comprises:
determining, based on a most recently generated measurement value in the plurality of measurement values of the blood glucose, whether the most recently generated measurement value is within the target range, below the lower limit or above the upper limit, wherein the most recently generated measurement value was generated after a most recently applied dose value in the plurality of dose values of the first type of insulin was applied; and
determining the next dose value of the first type of insulin based on the determining of whether the most recently generated measurement value is within the target range, below the lower limit or above the upper limit in accordance with the plurality of dose adjustment rules.

16. The computer-implemented method of claim 15, wherein determining the next dose value of the first type of insulin based on the determining of whether the most recently generated measurement value is within the target range, below the lower limit or above the upper limit comprises:
in response to the determining that the most recently generated measurement value is within the target range, setting the most recently applied dose value of the first type of insulin as the next dose value of the first type of insulin;
in response to the determining that the most recently generated measurement value is above the upper limit of the target range, determining the next dose value of the first type of insulin by increasing the most recently applied dose value of the first type of insulin by a predetermined amount of insulin or by a predetermined percentage of the most recently applied dose value; or
in response to the determining that the most recently generated measurement value is below the lower limit of the target range, determining the next dose value of the first type of insulin by decreasing the most recently applied dose value of the first type of insulin by a predetermined amount of the first type of insulin or by a predetermined percentage of the most recently applied dose value.

17. The computer-implemented method of claim 10, wherein the method further comprises:
determining whether a hypoglycemic event has occurred based on the plurality of measurement values; and
in response to the determining that the hypoglycemic event has occurred, causing a display, on the user interface, of a prompt to the patient to contact a healthcare provider.

18. One or more non-transitory computer-readable storage media coupled to one or more processors and having instructions stored thereon which, when executed by the one or more processors, cause the one or more processors to perform operations comprising:
retrieving, from a data storage, input data comprising:
a medical prescription specifying a dose plan for stabilizing a blood glucose level of a patient, the dose plan comprising (i) a plurality of dose adjustment rules for adjusting a dose of a first type of insulin, and (ii) a dose maintenance rule for maintaining a dose of a second type of insulin,
a plurality of measurement values of the blood glucose level generated by a blood glucose measurement unit, and
a plurality of dose values of the first type of insulin applied before the plurality of measurement values are generated; and
determining a next dose value of the first type of insulin based on the plurality of measurement values of the blood glucose level, the plurality of dose values of the first type of insulin, and the plurality of dose adjustment rules,
causing a display, on a user interface of a computing device, of the next dose value of the first type of insulin,
determining a next dose value of the second type of insulin based on the dose maintenance rule, and
causing a display, on the user interface, of the next dose value of the second type of insulin.

19. The one or more non-transitory computer-readable storage media of claim 18, wherein the first type of insulin is basal insulin and the second type of insulin is bolus insulin.

20. The one or more non-transitory computer-readable storage media of claim 18, wherein the first type of insulin is bolus insulin and the second type of insulin is basal insulin.
